(19) United States
(12) Patent Application Publication
(10) Pub. No.: US 2003/0190745 A1
(43) Pub. Date: Oct. 9, 2003

(54) Disk testing apparatus

(76) Inventors: R. Keith Galloway, Fairfax Station, VA (US); Steven S. Bachand, Dana Point, CA (US)

Correspondence Address: WALTER A. HACKLER, 2372 S.E. BRISTOL, SUITE B, NEWPORT BEACH, CA, 92660-0755, US

(21) Appl. No.: 10/118,832

(22) Filed: Apr. 8, 2002

(51) Int. Cl. C12M 1/34; G01N 33/543

(52) U.S. Cl. 435/287.2; 436/518

(57) ABSTRACT

A drug testing urine collection cup includes a cup body having a top bottom flat sidewall with the flat sidewall being transparent. At least one test strip is provided for assaying a fluid sample and visually displaying an assay result. A disk sized for insertion into the cup top for suspending a test strip into the cup body proximate the flat sidewall at a distance enabling visual perception of the assay through the transparent flat sidewall. A cap is provided for sealing the cup top for enabling transport thereof. In addition, the cap may engage the disk for removal of the disk from the cup if such action is desired. Alternatively, the disk may be left in the cap after easy removal of the test strip, the disk then enabling sealing of the cap to the cup for shipping to a confirmation testing site.

Present invention generally relates to diagnostic testing devices and more particularly is directed to a cup for containing a fluid sample, for example a urine specimen, and an insertion disk for providing indication of characteristics of the fluid specimen.

Fluid specimens, particularly body fluid such as urine, are usually collected and stored in cups or the like. Typically, cup is sealed with a lid which is thereafter punctured or removed in order to transfer samples to a separate test apparatus.

During this procedure fluids can escape and cause contamination these user processing errors may also contribute to incorrect results.

Many devices have been developed and are commercially available for the storage and testing fluid samples however such devices do not provide simplicity and convenience for their use. As an example, U.S. Pat. No. 5,976,895 describes a urine-testing cup that has a slotted cap in which the user must perform multiple manipulations for use. Sealing of the cup after testing requires even more manipulation.

U.S. Pat. No. 5,916,815 describes another type of cup that requires vigorous shaking to run the test and U.S. Pat. No. 5,403,551 requires positioning the cap in the defined location, tipping the cup to a specific angle for a set period of time and then waiting until a test valid signal appears in order to interpret the test.

None of these or other heretofore developed test cup devices represent a true one-step process. In the present invention, disk testing apparatus is introduced into previously collected fluid specimens within a cup and the result is read without further manipulation.

### SUMMARY OF THE INVENTION

A drug testing collection cup in accordance with the present invention generally includes a cup body having a top, bottom and a flat sidewall with the flat sidewall being transparent.

At least one test strip is provided for assaying a fluid specimen, such as, for example, urine, and visually displaying an assay result.

A disk is provided and sized for insertion into the cup for suspending the test strip within the cup proximate the flat sidewall at a distance enabling a visual perception of the assay result to the transparent flat sidewall.

A cap is provided for sealing the cup top to enable safe storage and transport thereof. Because of the proximity of the test strip to the transparent sidewalls, easy determination of the test results are determined without opening the cup. In addition, no agitation, manipulation or any other handling is required in order to effect an assay of the fluid specimen within the cup.

More particularly, the flat sidewall extends from the cup bottom to a point proximate the cup top and the strip is disposed proximately parallel to the flat sidewall. This structural configuration enables a plurality of relatively long test strips to be utilized and observed through the flat of viewing surface of the sidewall.

Still more particularly, the disk includes at least one member, depending from a bottom of the disk, for releasably grabbing a test strip. A plurality of strips may be supported by the disk and may be, if desired, permanently attached to the disk. Preferably, however, the member for grasping the test strip enables test strips of different configuration to be utilized with the present invention with the final selection of particular assay strips being made by the user.

To facilitate the handling of the disk a finger grip is disposed on the top of the disk for enabling manual insertion and removal of the disk, if desired, from the cup.

Preferably, the cup top is circular and includes threads therein for releasably engaging the cap. This configuration enables a fluid seal to be established. In addition, the disk may include a circular perimeter for facilitating its introduction to the cup by a user.

The cap may include means for engaging a disk upon sealing of the cap to the cup which enables removal of the disk with the cap as the cap is removed from the cup. This embodiment is preferred when storage or shipping of the cup is to be done without the test strips therein. In this case, a second cap is provided for sealing the cup after removal of the first cap and the disk from the cup. Alternatively, the test strip may be easily removed (pulled out) from the disk, leaving the disk in the cap. The cap/disk assembly can then be placed on the cup and turned to seal the cup for shipping. The disk has a thin sealing surface on its perimeter that acts as a gasket between the cap and cup.

### BRIEF DESCRIPTION OF THE DRAWINGS

The advantages and features of the present invention will be better understood by the following description when considered in conjunction with the accompanying drawings in which:

FIG. 1 is a perspective view of the drug testing collection cup in accordance with the present invention generally showing a cup having a flat side along with a disk positioned thereon;

FIG. 2 is a view similar to FIG. 1 illustrating a transparent cup and the positioning of a drug strip within the cup by the disk;

FIG. 3 is a side view of the apparatus of FIGS. 1 and 2 shown in cross section along with a cap for sealing the cup;

FIG. 4 is a side view of the disk in accordance with the present invention showing a means for removably grasping the test strips or cards and a finger grip for facilitating handling of the disk;

FIG. 5 is a perspective top view of the disk showing in FIG. 4;

FIG. 6 is a perspective bottom view of the disk showing in FIG. 4; and

FIG. 7 is a bottom perspective view of the disk engaged with the cap to facilitate its removal from the cup.

### DETAILED DESCRIPTION

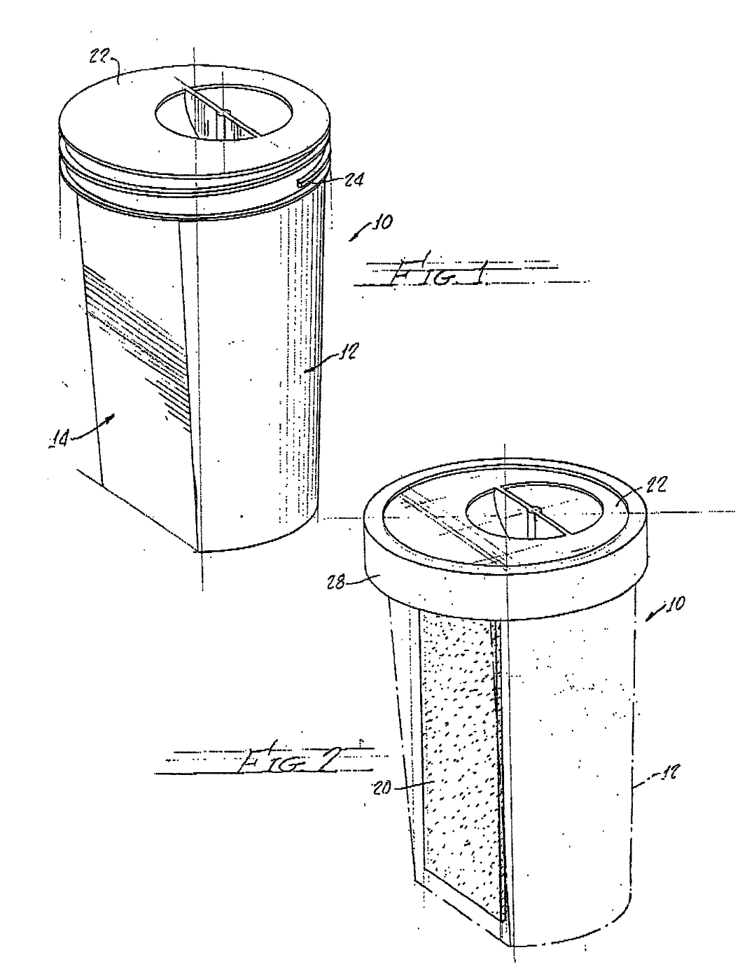

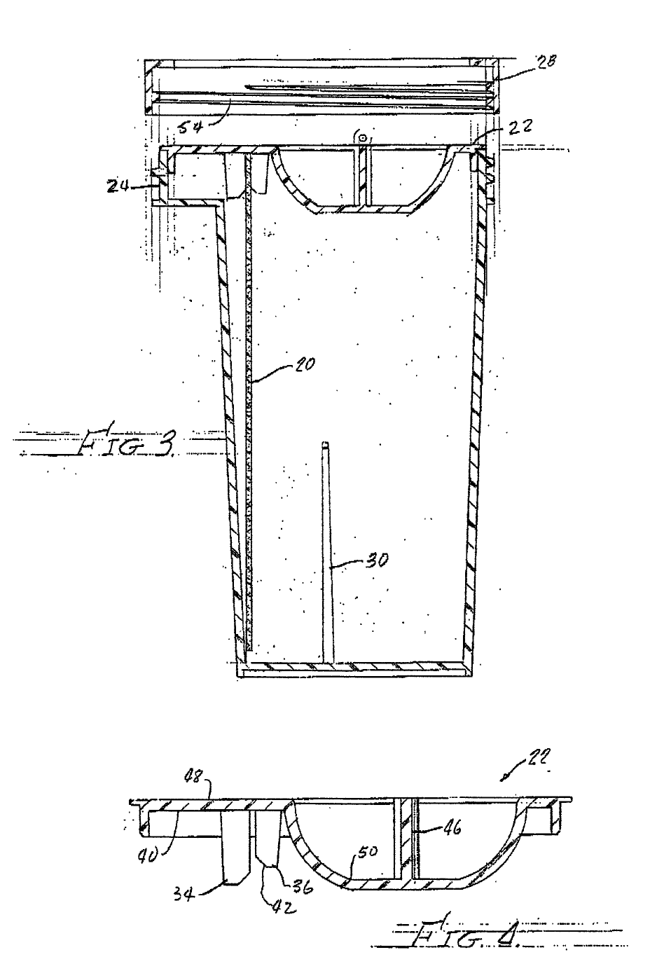

With reference to FIGS. 1-3 there is shown a drug testing collection cup 10 in accordance with the present invention which generally includes a cup body 12 which includes a flat sidewall 14 which is transparent. This provides a clear flat viewing configuration of a test strip or card 20 as shown in FIGS. 2 and 3.

A disk 22, see also FIG. 4, is provided and size for insertion into a cup top 24, which suspends the strip 20 within the cup body 12 proximate the flat clear sidewall 14 at a distance enabling visual perception of the assay result exhibited by the test strip through the sidewall 14.

The cup 12 disk 22 and cap 28, see FIG. 3, may be formed from any suitable material such as, for example but not limited to, polystyrene, polyethylene or polypropylene. The strip 20 may be of any suitable manufacturer for assaying a fluid sample, such as urine, and visually displaying an assay result.

Ribs 30 may be provided for preventing complete nesting of the cups 10 when stacked, one inside another, in order that locking of cups 10 to one another does not occur. Such stacking, enabled by the cup 10 configuration is preferable for storage and shipping considerations.

Rigidity of the sidewall 14 enables a spacing between the card or strip 20 and the sidewall of between about 0.10 inches and about 0.20 inches. It should be appreciated that these dimensions are referenced to reflect actual design and it should be understood that other spacing distances may also be appropriate.

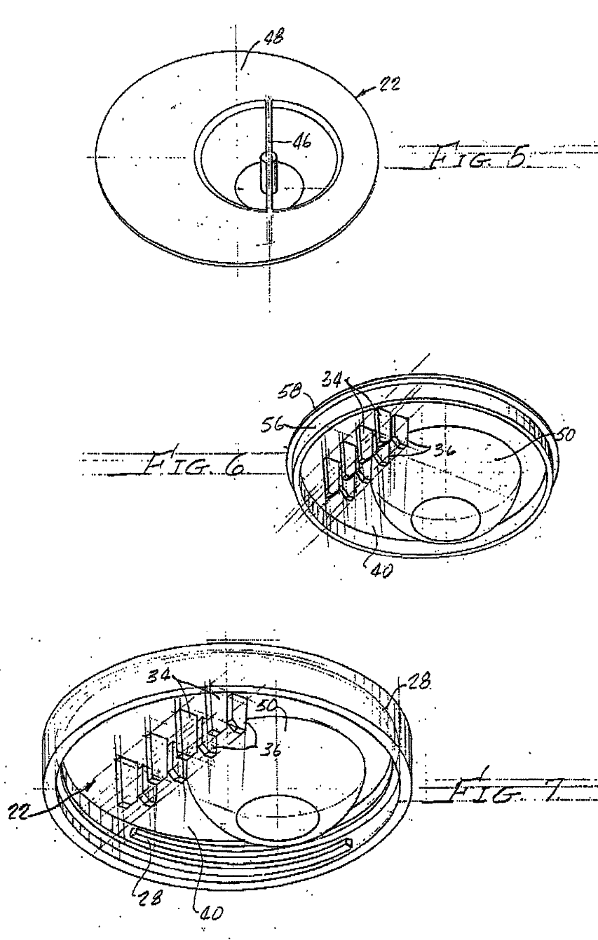

With particular reference to FIG. 4 the disk 22 includes members 34, 36 which depend from an underside 40 of the disk and are preferably molded therein at a spaced apart distance for providing a gap 42 therebetween for receiving the strip, or card, 20, resiliency of the members 34, 36 enabling a pressure fit against the strip 20 enabling removal and adjustment of the strip 20 or strips in a parallel array. Also as shown in FIGS. 4-7 the disk 22 includes a finger grip 46 molded into a top 48 of the disk.

A depending well 50 provides for clearance for finger engagement with the grip 46. This arrangement enables handling of the disk while at the same time no portion protrudes above the disk top 48.

The cup body 12 has a circular top 24 which enables the cap 28 with threads 54 to be screwed thereon in order to seal the cup top 24. The disk 22 also preferably includes a circular perimeter and a ridge 58 that serves as a perimeter flange for sealing the surface between the cup, extending therefrom which provides a means for engaging the cap 28 upon screwing of the cap 28 onto the cup top 24. The engagement is caused by the forcing of the ridge 28 past the threads in the cap as shown in FIG. 7.

Thus the strip 20 may be removed from the cup 12 by unscrewing of the cap 28 with the disk with the 28 attached thereto. Flexibility of the disk 22 enables the rotation thereof within the cap 28 and cup body 12 during strip 20 removal.

The cup 12 is thereafter sealed with a second cap, which may be identical to that shown in the Figures, and indicated with the character reference 28. Alternatively, after removing the strip 20 the disk 22 may be left in the cap 28 with the thin perimeter ridge or flange 58 acting as a seal.

While a ridge 58 and thread 54 provide a means for engaging the disk 22 and cap 28 it should be appreciated that any other suitable means for enabling engagement of the disk 22 and cap 28 are to be considered within the scope of the present invention.

However, the ridge 28 and cap threads 54 provide a simple economical means for engagement which may not be provided by other engagement devices, not shown.

Although there has been hereinabove described a drug testing collection in accordance with the present invention for the purpose of illustrating the manner in which the invention may be used to advantage, it should be appreciated that the invention is not limited thereto. Accordingly, any and all modifications, variations, or equivalent arrangements which may occur to those skilled in the arts should be considered within the scope of the present invention as defined in the appended claims.

What is claimed is:

1. A drug testing urine collection cup comprising:
a cup body having a top, bottom and a flat sidewall, said flat sidewall being transparent;
at least one test strip for assaying a urine sample and visually displaying an assay result;
a disk, sized for insertion into the cup top, for suspending the test strip within the cup body proximate the flat sidewall at a distance enabling visual perception of the assay result through the transparent flat sidewall; and
a cap for sealing the cup top for enabling transport thereof.

2. The collection cup according to claim 1 wherein the flat sidewall extends from the cup bottom to a point proximate the cup top and the strip is disposed approximately parallel to the flat sidewall.

3. The collection cup according to claim 2 wherein the cup top is circular and includes thread thereon for releasably engaging said cap.

4. The collection cup according to claim 3 wherein said disk includes at least one member, depending from a bottom of said disk for releasing grasping the test strip.

5. The collection cup according to claim 4 wherein said disk includes a finger grip, disposed on a top of said disk for enabling manual insertion and removal of said disk into and out of said cup.

6. The collection cup according to claim 5 wherein at least two members, depending from the disk bottom, are adjacently disposed for enabling press-fit grasping of the test strip.

7. The collection cup according to claim 3 wherein the disk includes a circular perimeter for providing a seal between said cap and said disk.

8. The collection cup according to claim 6 further comprising means for engaging said disk and cap upon sealing of the cap to said cup, for enabling removal of said disk with said cap as said cap is removed from said cup.

9. The collection cup assembly according to claim 8 wherein the means for engaging the disk and cap includes a flange formed in the disk and threads formed in the cap.

10. A drug testing collection cup comprising:
a cup body having a top, bottom and a flat sidewall, said flat sidewall being transparent;
at least one test strip for assaying a fluid sample and visually displaying an assay result;
a disk, sized for insertion into the cup top, for suspending the test strip within the cup body proximate the flat sidewall at a distance enabling visual perception of the assay results through the transparent flat sidewall; and
a first cap for sealing the cup top and engaging said disk, the engagement enabling removal of said disk, and test strip, from said cup; and
a second cap for sealing the cup top for enabling transport thereof.

11. The collection cup according to claim 10 wherein the flat sidewall extends from the cup bottom to a point proximate the cup top and the strip is disposed approximate parallel to the flat sidewall.

12. The collection cup according to claim 11 wherein the cup top is circular and includes threads thereon for releasably engaging the first and second cap.

13. The collection cup according to claim 12 wherein said disk includes at least one member, depending from a bottom of said disk for releasing grasping the test strip.

14. The collection cup according to claim 13 wherein said disk includes a finger grip, disposed on a top of said disk for enabling manual insertion of said disk into said cup.

15. The collection cup according to claim 14 wherein at least two members, depending from the disk bottom, are adjacently disposed for enabling press-fit grasping of the test strip.

16. The collection cup according to claim 12 wherein the disk includes a circular perimeter for sealing said cap to said cup.

17. The collection cup according to claim 16 wherein the disk includes a perimeter flange for engaging threads in said cup for providing engagement therebetween
